SEQUENCE LISTING (2 sequences)

SEQ ID NO: 1 - DNA, 51 bp, Homo sapiens
  source 1..51: mol_type = unassigned DNA
atcattggctgacactttcgaacacrtgatagaagagctgttggatgagga

SEQ ID NO: 2 - DNA, 1458 bp, Homo sapiens
  source 1..1458: mol_type = unassigned DNA
ctgcagaaaggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacrtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgcacccccaaggggaactaaccgcctcccacacactatattcctgccacccccgccccaccctacaccggccccgcaccgcccc

(19) United States
(12) Patent Application Publication
(10) Pub. No.: US 2025/0255835 A1
(43) Pub. Date: Aug. 14, 2025

(54) Methods For The Treatment Of Depression

(71) Applicant: Janssen Pharmaceutica NV, Beerse (BE)

(72) Inventors: Lodewijk Ivo Caers, Beerse (BE); Jaskaran Singh, San Diego, CA (US); Peter Nicholas Zannikos, Doylestown, PA (US); Wayne C. Drevets, Rancho Santa Fe, CA (US); Ella Daly, Doylestown, PA (US); Carla Maria Canuso, Yardley, PA (US); Margaret Fedgchin, Collegeville, PA (US); Frank Wiegand, Annandale, NJ (US); Giacomo Salvadore, Philadelphia, PA (US)

(21) Appl. No.: 19/030,701

(22) Filed: Jan. 17, 2025

Related U.S. Application Data
(63) Continuation of application No. 17/726,931, filed on Apr. 22, 2022.
Application No. 17/726,931 is a continuation of application No. 17/345,395, filed on Jun. 11, 2021, now Pat. No. 11,311,500.
Application No. 17/345,395 is a continuation of application No. 17/129,508, filed on Dec. 21, 2020, now Pat. No. 11,173,134.
Application No. 17/129,508 is a continuation of application No. 16/727,594, filed on Dec. 26, 2019, now Pat. No. 10,869,844.
Application No. 16/727,594 is a continuation of application No. 14/853,351, filed on Sep. 14, 2015, now abandoned.
(60) Provisional application No. 62/050,439, filed on Sep. 15, 2014.

(51) Int. Cl. A61K 31/135; A61K 9/00; C12Q 1/6883

(52) U.S. Cl. CPC A61K 31/135; A61K 9/0043; C12Q 1/6883; C12Q 2600/106; C12Q 2600/156

(57) ABSTRACT

The present invention is directed to methods and dosing regimens for the treatment of depression (preferably, treatment resistant depression), for the treatment of depression in a suicidal patient, and/or for the treatment and/or prevention of suicidality (e.g. suicidal ideations).

### CROSS REFERENCE TO RELATED APPLICATIONS

This application is a continuation of U.S. patent application Ser. No. 17/726,931, filed Apr. 22, 2022, which is a continuation of U.S. patent application Ser. No. 17/345,395, filed Jun. 11, 2021, which is a continuation of U.S. patent application Ser. No. 17/129,508, filed Dec. 21, 2020, which is a continuation of U.S. patent application Ser. No. 16/727,594, filed Dec. 26, 2019, which is a continuation of U.S. patent application Ser. No. 14/853,351, filed Sep. 14, 2015, which claims the benefit of priority under 35 U.S.C. § 119 (e) to U.S. Provisional Patent Application 62/050,439, filed Sep. 15, 2014, the disclosures of which are herein incorporated by reference in their entireties.

### SEQUENCE LISTING

The instant application contains a Sequence Listing which has been submitted electronically in XML format and is hereby incorporated by reference in its entirety. Said XML copy, created on Apr. 30, 2025, is named PRD3353USCNT5-103693_4337_SL.xml and is 4,286 bytes in size.

### FIELD OF THE INVENTION

The present invention is directed to methods and dosing regimens for the treatment of depression (preferably treatment resistant depression), for the treatment of depression in a suicidal patient, and/or for the treatment and/or prevention of suicidality (e.g. suicidal ideations) comprising genotyping a patient to determine their Val66Met rs6265 polymorphism in BDNF and administering a ketamine, preferably esketamine, preferably intranasal esketamine, according to a dosing regimen matched to the patient's genotype.

### BACKGROUND OF THE INVENTION

Major Depressive Disorder is defined as the presence of one of more major depressive episodes that are not better accounted for psychotic disorder or bipolar disorder. A major depressive episode is characterized by meeting five or more of the following criteria during the same 2 week period which represent a change in functioning and include at least depressed/sad mood or loss of interest and pleasure, indifference or apathy, or irritability and is usually associated with a change in a number of neurovegetative functions, including sleep patterns, appetite and body weight, motor agitation or retardation, fatigue, impairment in concentration and decision making, feelings of shame or guilt, and thoughts of death or dying (Harrison's Principles of Internal Medicine, 2000). Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning. (Diagnostic and Statistical Manual of Mental Disorders, 4^{th} Edition, American Psychiatric Association, 1994)

Current treatment options for unipolar depression include monotherapy or combination therapy with various classes of drugs including mono-amine oxidase inhibitors (MAOI), tricyclic antidepressants (TCA), serotonin specific reuptake inhibitors (SSRI), serotonin noradrenergic reuptake inhibitors (SNRI), noradrenaline reuptake inhibitor (NRI), “natural products” (such as Kava-Kava, St. John's Wort), dietary supplement (such as s-adenosylmethionine) and others. More specifically, drugs used in the treatment of depression include, but are not limited to imipramine, amitriptyline, desipramine, nortriptyline, doxepin, protriptyline, trimipramine, maprotiline, amoxapine, trazodone, bupropion, chlomipramine, fluoxetine, citalopram, sertraline, paroxetine, tianeptine, nefazadone, venlafaxine, desvenlafaxine, duloxetine, reboxetine, mirtazapine, phenelzine, tranylcypromine, and/or moclobemide. Several of these agents including, but not limited to, serotonin reuptake inhibitors are also used when depression and anxiety co-exist, such as in anxious depression.

In the clinic, 40-50% of depressed patients who are initially prescribed antidepressant therapy do not experience a timely remission of depression symptoms. This group typifies level 1 treatment-resistant depression, that is, a failure to demonstrate an “adequate” response to an “adequate” treatment trial (that is, sufficient intensity of treatment for sufficient duration). Moreover, about approximately 30% of depressed patients remain partially or totally treatment-resistant to at least two antidepressant treatments including combination treatments. Increasingly, treatment of treatment-resistant depression includes augmentation strategies including treatment with pharmacological agents such as, antipsychotics (such as quetiapine, aripiprazole, olanzapine, risperidone, and the like), lithium, carbamazepine, and triiodothyronine, and the like; adjunctive electroconvulsive therapy; adjunctive transcranial magnetic stimulation; etc.

Suicide, also known as completed suicide, is the “act of taking one's own life”. Attempted suicide or non-fatal suicidal behavior is self-injury with the desire to end one's life that does not result in death. Suicidal ideation is the medical term for thoughts about or an unusual preoccupation with suicide, or thoughts of ending one's life or not wanting to live anymore but not necessarily taking any active efforts to do so.

The range of suicidal ideation varies greatly from fleeting to chronic and progress to detailed planning, role playing, and unsuccessful attempts, which may be deliberately constructed to fail or be discovered, or may be fully intended to result in death. Although not all who have suicidal ideation go on to make suicide attempts, a significant proportion do. Suicidal ideation is generally associated with depression (at about 60-70% of all cases).

Suicidal ideation which may include, for example, suicidal thoughts, may also include other related signs and symptoms. Some symptoms or co-morbid conditions may include unintentional weight loss, feeling helpless, feeling alone, excessive fatigue, low self-esteem, presence of consistent mania, excessively talkative, intent on previously dormant goals, feel like one's mind is racing. The onset of symptoms like these with an inability to get rid of or cope with their effects, a possible form of psychological inflexibility, is one possible trait associated with suicidal ideation. They may also cause psychological distress, which is another symptom associated with suicidal ideation. Symptoms like these related with psychological inflexibility, recurring patterns, or psychological distress may in some cases lead to the onset of suicidal ideation. Other possible symptoms and warning signs include: hopelessness, anhedonia, insomnia, depression, severe anxiety, angst, impaired concentration, psychomotor agitation, panic attack and severe remorse.

Scales used in the evaluation of suicidal ideation include Beck Scale for Suicide Ideation (BSS), Columbia Suicide Severity Rating Scale (C-SSRS), Suicidal Ideation and Behavioral Assessment Tool (SIBAT) and The Kessler Psychological Distress Scale (K10, which test does not measure suicidal ideation directly, but there may be value in its administration as an early identifier of suicidal ideation. High scores of psychological distress are also, in some cases associated with suicidal ideation.

There are also several psychiatric disorders that appear to be comorbid with suicidal ideation or considerably increase the risk of suicidal ideation. The following disorders have been shown to be the strongest predictors of suicidal ideation/disorders in which risk is increased to the greatest extent: major depressive disorder (MDD), dysthymia, bipolar disorder. The main treatments for suicidality and/or suicidal ideation include: hospitalization, outpatient treatment, and medication. Hospitalization allows the patient to be in a secure, supervised environment to prevent their suicidal ideation from turning into suicide attempts. In most cases, individuals have the freedom to choose which treatment they see fit for themselves. However, there are several circumstances in which individuals can be hospitalized involuntarily, per state law including circumstances where an individual poses danger to self or others and where an individual is unable to care for one's self.

Outpatient treatment allows individuals to remain at their place of residence and receive treatment when needed or on a scheduled basis. Before allowing patients the freedom that comes with outpatient treatment, physicians evaluate several factors of the patient. These factors include the patient's level of social support, impulse control and quality of judgment. After the patient passes the evaluation, they are often asked to consent to a “no-harm contract”. This is a contract formulated by the physician and the family of the patient. Within the contract, the patient agrees not to harm themselves, to continue their visits with the physician, and to contact the physician in times of need. These patients are then checked on routinely to assure they are maintaining their contract and staying out of troublesome activities.

There are also a number of different pharmacological treatment options for those experiencing suicidal ideation. However, prescribing medication to treat suicidal ideation can be difficult. One reason for this is because many medications lift patients' energy levels before lifting their mood. This puts them at greater risk of following through with attempting suicide. Additionally, if a patient has a co-morbid psychiatric disorder, it may be difficult to find a medication that addresses both the psychiatric disorder and suicidal ideation. Therefore, the medication prescribed to one suicidal ideation patient may be completely different than the medication prescribed to another patient. Although research is largely in favor of the use of antidepressants for the treatment of suicidal ideation associated with depression, in some cases antidepressants are claimed to be associated with increased suicidal ideation. Upon the start of using antidepressants, many clinicians will note that sometimes the sudden onset of suicidal ideation may accompany treatment. This has caused the Food and Drug Administration (FDA) to issue a warning stating that sometimes the use of antidepressants may actually increase the thoughts of suicidal ideation.

Ketamine (a racemic mixture of the corresponding S- and R-enantiomers) is an NMDA receptor antagonist, with a wide range of effects in humans, depending on the dose, including for example, analgesia, anesthesia, hallucinations, dissociative effects, elevated blood pressure and bronchodilation. Ketamine is primarily used for the induction and maintenance of general anesthesia. Other uses include sedation in intensive care, analgesia (particularly in emergency medicine and treatment of bronchospasms. Ketamine has also been shown to be efficacious in the treatment of depression (particularly in those who have not responded to other anti-depressant treatment). In patients with major depressive disorders, ketamine has additionally been shown to produce a rapid antidepressant effect, acting within two hours.

The S-ketamine enantiomer (or S-(+)-ketamine or esketamine) has higher potency or affinity for the NMDA reception and thus potentially allowing for lower dosages; and is available for medical use under the brand name KETANEST S in some countries.

Brain-derived neurotropic factor (BDNF) is a secreted protein that, in humans, is encoded by the BDNF gene. BDNF is a member of the “neurotrophin” family of growth factors, which are related to the canonical “nerve growth factor”, NGF. BDNF acts on certain neurons of the central nervous system (CNS) and the peripheral nervous system (PNS), helping to support the survival of existing neurons, and encourage the growth and differentiation of new neurons and synapses. In the brain, it is active in the hippocampus, cerebral cortex, and basal forebrain-areas vital to learning, memory, and higher thinking. BDNF is also is important for long-term memory. The BDNF protein is coded by the gene that is also called BDNF. In humans this gene is located on chromosome 11. Val66Met (rs6265) is a single nucleotide polymorphism in the gene where adenine and guanine alleles vary, resulting in a variation between valine (Val) and methione (Met) at codon 66.

A decrease in brain-derived neurotrophic factor (BDNF) expression in medial prefrontal cortex (mPFC) and other regions has given rise to the BDNF hypothesis of major depression. The human polymorphism in the BDNF gene, which leads to a valine-to-methionine substitution in the proBDNF protein at codon 66 (Val66Met), is carried by approximately 30% of the general Caucasian population (and approximately by 60-80% of the Asian population) and has been associated with mild cognitive deficits and possibly decreased hippocampal volume. In addition, individuals who carry the Met polymorphism have been reported to have an increased risk of stress-related major depression. The Val66Met polymorphism impairs activity dependent secretion of BDNF at synaptic sites and reduces intracellular trafficking of BDNF messenger RNA (mRNA) to dendrites.

The Val66Met polymorphism of BDNF has been registered in the dbSNP as r$6265 [Homo sapiens], occurring on the 11:27658369 chromosome and defined by the following sequence, SEQ. ID. No. 1:

ATCATTGGCTGACACTTTCGAACAC[A/G]TGATAGAAGAGCTGTTGGA
TGAGGA

where [A/G] defines the position of the mutation.

Standard antidepressants, electroconvulsive therapy, and brain stimulation techniques such as transcranial magnetic stimulation all increase peripheral BDNF levels; also exercise has BDNF secretion-enhancing effects.

In a letter to the editor published in the peer-reviewed journal Biological Psychiatry, LAJE, G., Biol. Psychiatry, 2012 report on an analysis of the effect of rs6265 (Val66Met SNP) on the response to ketamine in patients experiencing a major depressive episode. The results “suggest that major depressive disorder (MDD) patients with the Val/Val BDNF allele at rs6265 are more likely to exhibit antidepressant response to ketamine than Met carriers.” This effect was initially hinted at by LIU, R-Y., Biol. Psychiatry, 2012, pp 996-1005, Vol. 71, who also suggested that Met/Met knocked in mice displayed blunted antidepressant-like response to ketamine compared to Val/Val mice, as well as reduced synaptogenesis (LIU, R-Y., et al.). Altogether, this evidence further suggests that determining the BDNF rs6265 genotype might be associated with likelihood of response to ketamine and that it may be possible to separate subjects with a higher likelihood of response to ketamine based on this genotype.

There remains a need to provide an effective treatment for depression, more particularly treatment resistant depression and/or for the treatment of suicidality, suicidal ideations, and for the prevention of suicide, particularly in the first hours and days after the onset of highly suicidal ideation, thoughts.

### SUMMARY OF THE INVENTION

The present invention is directed to a method for the treatment of depression (preferably treatment resistant depression), for the treatment of depression in a suicidal patient, and/or for the treatment and/or prevention of suicidality (e.g. suicidal ideations) comprising
Step A: genetically testing (or genotyping) a patient suffering from depression to determine their Val66Met rs6265 polymorphism in the BDNF gene (i.e. to determine if the patient is a Val/Val homozygote, a Val/Met heterozygote or a Met/Met homozygote); and
Step B: administering an esketamine dosing regimen; wherein the esketamine is preferably administered intranasally; and wherein the dosing regimen comprises
   (i) an induction dosing phase;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosing frequency of one to five times per week (preferably, one to three times per week, preferably once or twice per week, preferably twice per week);
   wherein, if the patient is a Val/Val homozygote, then the esketamine is administered at a dosage in an amount in the range of from about 28 mg to about 56 mg;
   wherein, if the patient is a Val/Met heterozygote or a Met/Met homozygote, then the esketamine is administered at a dosage in an amount in the range of from about 56 mg to about 84 mg;
   and wherein, during the induction phase, the dosage amount and/or the dosing frequency for the patient who is a Val/Val homozygote and the dosage amount and/or dosing frequency for the patient who is a Val/Met heterozygote or Met/Met homozygote (i.e. the patient is a Met carrier) are different; (preferably, the dosage amount is different);
   and (b) a maintenance phase;
   wherein the maintenance phase comprises a treatment period of at least 6 weeks (preferably at least 8 weeks, preferably at least 10 weeks, more preferably at least 12 weeks, more preferably at least 14 weeks);
   wherein, if the patient is a Val/Val homozygote, then the esketamine is administered at a dosage in an amount in the range of from about 28 mg to about 56 mg; and wherein the esketamine is administered at a dosing frequency in the range of once every two weeks to once every four weeks;
   wherein, if the patient is a Val/Met heterozygote or a Met/Met homozygote, then the esketamine is administered at a dosage in an amount in the range of from about 56 mg to about 84 mg; and wherein the esketamine is administered at a dosing frequency in the range of once per week to once every two weeks;
   and wherein, during the maintenance phase, the dosage amount and/or the dosing frequency for the patient who is a Val/Val homozygote and the dosage amount and/or dosing frequency for the patient who is a Val/Met heterozygote or Met/Met homozygote (i.e. the patient is a Met carrier) are different; (preferably, the dosage amount is different);
   and wherein the maintenance phase preferably continues until further treatment is not required (as determined by a clinician or physician).

The present invention is further directed to a method for the treatment of depression (preferably treatment resistant depression), for the treatment of depression in a suicidal patient, and/or for the treatment and/or prevention of suicidality (e.g. suicidal ideations) comprising
Step A: genetically testing (or genotyping) a patient suffering from depression to determine their Val66Met rs6265 polymorphism in the BDNF gene (i.e. to determine if the patient is a Val/Val homozygote, a Val/Met heterozygote or a Met/Met homozygote); and
Step B: administering esketamine, preferably intranasally, according to an induction phase regimen;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosing frequency of one to five times per week (preferably, one to three times per week, preferably once or twice per week, preferably twice per week);
   wherein, if the patient is a Val/Val homozygote, then the esketamine is administered at a dosage in an amount in the range of from about 28 mg to about 56 mg;
   wherein, if the patient is a Val/Met heterozygote or a Met/Met homozygote, then the esketamine is administered at a dosage in an amount in the range of from about 56 mg to about 84 mg;
   and wherein, during the induction phase, the dosage amount and/or the dosing frequency for the patient who is a Val/Val homozygote and the dosage amount and/or dosing frequency for the patient who is a Val/Met heterozygote or Met/Met homozygote (i.e. the patient is a Met carrier) are different; (preferably, the dosage amount is different).

The present invention is further directed to a method for the treatment of depression (preferably treatment resistant depression), for the treatment of depression in a suicidal patient, and/or for the treatment and/or prevention of suicidality (e.g. suicidal ideations) comprising
Step A: genetically testing (or genotyping) a patient suffering from depression to determine their Val66Met rs6265 polymorphism in the BDNF gene (i.e. to determine if the patient is a Val/Val homozygote, a Val/Met heterozygote or a Met/Met homozygote); and
Step B: administering esketamine, preferably intranasally, according to a maintenance phase regimen;
   wherein the maintenance phase comprises a treatment period of at least 6 weeks (preferably at least 8 weeks, preferably at least 10 weeks, more preferably at least 12 weeks, more preferably at least 14 weeks);
   wherein, if the patient is a Val/Val homozygote, then the esketamine is administered at a dosage in an amount in the range of from about 28 mg to about 56 mg; and wherein the esketamine is administered at a dosing frequency in the range of once every two weeks to once every four weeks;
   wherein, if the patient is a Val/Met heterozygote or a Met/Met homozygote, then the esketamine is administered at a dosage in an amount in the range of from about 56 mg to about 84 mg; and wherein the esketamine is administered at a dosing frequency in the range of once per week to once every two weeks;
   wherein, during the maintenance phase, the dosage amount and/or the dosing frequency for the patient who is a Val/Val homozygote and the dosage amount and/or dosing frequency for the patient who is a Val/Met heterozygote or Met/Met homozygote (i.e. the patient is a Met carrier) are different; (preferably, the dosage amount is different);
   and wherein the maintenance phase preferably continues until further treatment is not required (as determined by a clinician or physician).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering an esketamine dosing regimen; wherein the esketamine is preferably administered intranasally; and wherein the dosing regimen comprises
   (i) an induction dosing phase;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosage in an amount in the range of from about 28 mg to about 56 mg;
   and wherein the esketamine is administered at a dosing frequency of one to five times per week (preferably, one to three times per week, preferably once or twice per week, preferably twice per week);
   and (b) a maintenance phase;
   wherein the maintenance phase comprises a treatment period of at least 6 weeks (preferably at least 8 weeks, preferably at least 10 weeks, more preferably at least 12 weeks, more preferably at least 14 weeks);
   wherein the esketamine is administered at a dosage in an amount in the range of from about 28 mg to about 56 mg;
   wherein the esketamine is administered at a dosing frequency in the range of once every two weeks to once every four weeks;
   and wherein the maintenance phase preferably continues until further treatment is not required (as determined by a clinician or physician).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering esketamine, preferably intranasally, according to an induction phase regimen;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosage in an amount in the range of from about 28 mg to about 56 mg;
   and wherein the esketamine is administered at a dosing frequency of one to five times per week (preferably, one to three times per week, preferably once or twice per week, preferably twice per week).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering esketamine, preferably intranasally, according to a maintenance phase regimen;
   wherein the maintenance phase comprises a treatment period of at least 6 weeks (preferably at least 8 weeks, preferably at least 10 weeks, more preferably at least 12 weeks, more preferably at least 14 weeks);
   wherein the esketamine is administered at a dosage in an amount in the range of from about 28 mg to about 56 mg;
   wherein the esketamine is administered at a dosing frequency in the range of once every two weeks to once every four weeks;
   and wherein the maintenance phase preferably continues until further treatment is not required (as determined by a clinician or physician).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering an esketamine dosing regimen; wherein the esketamine is preferably administered intranasally; and wherein the dosing regimen comprises
   (i) an induction dosing phase;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosage in an amount in the range of from about 56 mg to about 84 mg;
   wherein the esketamine is administered at a dosing frequency of one to five times per week (preferably, one to three times per week, preferably once or twice per week, preferably twice per week);
   and (b) a maintenance phase;
   wherein the maintenance phase comprises a treatment period of at least 6 weeks (preferably at least 8 weeks, preferably at least 10 weeks, more preferably at least 12 weeks, more preferably at least 14 weeks);
   wherein the esketamine is administered at a dosage in an amount in the range of from about 56 mg to about 84 mg;
   wherein the esketamine is administered at a dosing frequency in the range of once per week to once every two weeks;
   and wherein the maintenance phase preferably continues until further treatment is not required (as determined by a clinician or physician).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering esketamine, preferably intranasally, according to an induction phase regimen;
   wherein the induction phase comprises a treatment period of between 2 and 8 weeks (preferably between 2 and 6 weeks, preferably between 2 and 4 weeks, for example, for 2 weeks, for 3 weeks, for 4 weeks, for 6 weeks or for 8 weeks);
   wherein the esketamine is administered at a dosage in an amount in the range of from about 56 mg to about 84 mg;
   and wherein the esketamine is administered at a dosing frequency of one to five times per week (preferably, one to three times per week, preferably once or twice per week, preferably twice per week).

The present invention is further directed to a dosing regimen for the treatment of depression (preferably, treatment resistant depression), for the treatment of depression in a suicidal patient, and/or for the treatment and/or prevention of suicidality (e.g. suicidal ideations) comprising:
A) administering esketamine, preferably intranasally, according to a maintenance phase regimen;
   wherein the maintenance phase comprises a treatment period of at least 6 weeks (preferably at least 8 weeks, preferably at least 10 weeks, more preferably at least 12 weeks, more preferably at least 14 weeks);
   wherein the esketamine is administered at a dosage in an amount in the range of from about 56 mg to about 84 mg;
   wherein the esketamine is administered at a dosing frequency in the range of once per week to once every two weeks;
   and wherein the maintenance phase preferably continues until further treatment is not required (as determined by a clinician or physician).

In an embodiment of the present invention, the esketamine is administered at a dosage in the range of from about 28 mg to about 84 mg (for example at about 28 mg, at about 56 mg or at about 84 mg); and at the lowest frequency at which the antidepressant response is maintained.

In another embodiment of the present invention, in maintenance dosing or during the maintenance phase of the dosing regimen, the esketamine is administered at a dosage in the range of from about 28 mg to about 84 mg (for example at about 28 mg, at about 56 mg or at about 84 mg); and at the lowest frequency at which the antidepressant response is maintained.

### BRIEF DESCRIPTION OF THE FIGURES

FIG. 1 illustrates the mean changes in MADRS total score from baseline after the 1^{st} infusion by Val/Val or Met carriers and by placebo or active.

FIG. 2 illustrates the survival curves for the duration of response in esketamine responders per day 1 randomization after Day 17 by Val/Val or Met carriers.

FIG. 3 illustrates survival curves for duration of response by Val/Val or Met carriers in all responders to esketamine after Day 17.

FIG. 4 illustrates mean changes in MADRS total scores from baseline over time up to Day 15 by Val/Val or Met carriers and by ketamine or placebo.

FIG. 5 illustrates survival curves for the duration of response Day 29 and up to Day 44 by Val/Val or Met carriers in ketamine responders.

### DETAILED DESCRIPTION OF THE INVENTION

The present invention is directed to methods and dosing regimens for the treatment of depression (preferably treatment resistant depression), for the treatment of depression in a suicidal patient, and/or for the treatment and/or prevention of suicidality (e.g. suicidal ideations) comprising genotyping a patient in need thereof and administering ketamine, preferably esketamine, preferably intranasal esketamine, according to a dosing regimen which is selected (preferably optimized) for said patient, based on the subject's Val66Met rs6265 BDNF genotype, and as described in more detail herein.

One skilled in the art will recognize that the maintenance phase of the dosing regimens of the present invention will continue until further treatment is not required, for example as determined by a clinician, physician, psychiatrist, psychologist, or other suitable medical professional, and as indicated by for example, prolonged remission of the depression (including for example, the remission of one or more symptoms associated with the depression), social and/or occupational functional improvement(s) to normal or premorbid levels, or other known measures of depression.

One skilled in the art will further recognize that in the methods and dosing regimens of the present invention, the maintenance of the antidepressant response in a patient may be determine by for example, a clinician, physician, psychiatrist, psychologist, or other suitable medical professional. Additionally, maintenance of the antidepressant response may be established by for example, an absence of relapse of the depression (or one or more symptoms of the depression), an absence of the need for additional or alternate treatment(s) for the depression, an absence of the worsening of the depression, an absence of the need for hospitalization for a suicidal attempt or to prevent suicide, or, when evaluated by MADRS score, by maintenance of a MADRS score less than about 22 and/or the absence of a MADRS score above 22 for any continuous two week period.

As used herein, the term “depression” shall be defined to include major depressive disorder, unipolar depression, treatment resistant depression, depression with anxious distress, bipolar depression and dysthymia (also referred to as dysthymic disorder). Preferably, the depression is major depressive disorder, unipolar depression, treatment resistant depression, depression with anxious distress, or bipolar depression. More preferably, the depression is major depressive disorder, unipolar depression, treatment resistant depression and bipolar depression. More preferably, the depression is treatment-resistant depression.

In an embodiment, the present invention is directed to methods and dosing regimens for the treatment of depression in suicidal patients. One skilled in the art will recognize that the term “depression in suicidal patients” shall include any type of depression as herein defined, when diagnosed in a patient that also exhibits at least one symptom of suicidality, for example suicidal ideations and/or behaviors (e.g. intent, planning, etc.). Thus, “depression in suicidal patients” includes, but is not limited to, major depressive disorder in suicidal patients, unipolar depression in suicidal patients, treatment resistant depression in suicidal patients, depression with anxious distress in suicidal patients, bipolar depression in suicidal patients and dysthymia in suicidal patients. Preferably, the “depression in suicidal patients” is selected from the group consisting of major depressive disorder in suicidal patients, unipolar depression in suicidal patients and treatment resistant depression in suicidal patients. More preferably, the “depression in suicidal patients” is treatment resistant depression in suicidal patients.

As used herein, the term “treatment-refractory or treatment-resistant depression” and the abbreviation “TRD” shall be defined as a major depressive disorder that does not respond to a least two antidepressant regimens or treatments.

As used herein, unless otherwise noted, the term “antidepressant” shall mean any pharmaceutical agent which can be used to treat depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, escitolapram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; atypical antidepressants such as bupropion, and the like; natural products such as Kava-Kava, St. John's Wort, and the like; dietary supplements such as s-adenosyimethionine., and the like; and neuropeptides such as thyrotropin-releasing hormone and the like; compounds targeting neuropeptide receptors such as neurokinin receptor antagonists and the like; and hormones such as triiodothyronine, and the like. Preferably, the antidepressant is selected from the group consisting of fluoxetine, imipramine, bupropion, venlafaxine and sertaline.

Therapeutically effective dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http:/www.pdrel.com) or other sources.

As used herein the term “antipsychotic” includes, but is not limited to:
(a) typical or 1^{st} generation antipsychotics, such as phenothiazines (e.g., chlorpromazine, thioridazine, fluphenazine, perphenazine, trifluoperazine, levomepromazin), thioxanthenes (e.g., thiothixene, flupentixol), butyrophenones (e.g., haloperidol), dibenzoxazepines (e.g., loxapine), dihydroindolones (e.g., molindone), substituted benzamides (e.g., sulpride, amisulpride), and the like; and
(b) atypical or 2^{nd} generation antipsychotics, such as paliperidone, clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, brexpiprazole, nemonapride, SR-31742 (Sanofi), CX-516 (Cortex), SC-111 (Scotia), NE-100 (Taisho), and the like.

In an embodiment, the “atypical antipsychotic” is selected from the group consisting of aripiprazole, brexpiprazole, quetiapine, olanzapine, risperidone and paliperidone. In another embodiment, the atypical antipsychotic is selected from the group consisting of aripiprazole, quetiapine, olanzapine and risperidone; preferably, the atypical antipsychotic is selected from the group consisting of aripiprazole, quetiapine and olanzapine.

One skilled in the art will recognize that wherein the present invention is directed to methods or dosing regimens for the treatment and/or prevention of suicidality, said methods and dosing regimens include, but are not limited to, the prevention of suicidal ideations, suicidal behaviors, suicidal attempts and/or suicide.

As used herein, unless otherwise noted, the term “esketamine” shall mean the (S)-enantiomer of ketamine, as its corresponding hydrochloride salt, a compound of formula (I)

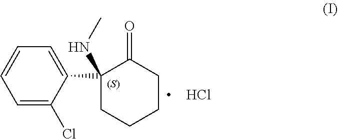

also known as (S)-2-(2-chlorophenyl)-2-(methylamino)cyclohexenone hydrochloride.

As used herein, unless otherwise noted, the term “Met carrier” shall mean a patient or subject who is either a Val/Met heterozygote or a Met/Met homozygote, as determined by testing said patient or subject for Val66Met rs6265 polymorphism in BDNF.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

As used herein, unless otherwise noted, the terms “subject” and “patient” refer to an animal, preferably a mammal, most preferably a human, who has been the object of treatment, observation or experiment. Preferably, the subject or patient has experienced and/or exhibited at least one symptom of the disease or disorder to be treated and/or prevented. One skilled in the art will further recognize that the methods of treatment or prevention and the dosing regimens of the present invention are directed to subjects or patients in need of such treatment, prevention or dosing regimen, more particularly to subjects or patients diagnosed with or exhibiting at least one symptom of depression (preferably, meeting the criteria for major depressive disorder or episode) regardless of type or underlying cause.

In an embodiment of the present invention, the subject or patient in need thereof is a subject or patient that has been diagnosed with or exhibits at least one symptom of depression (preferably, meeting the criteria for major depressive disorder or episode) and who has further been diagnosed with or exhibits at least one symptom of suicidality (e.g. suicidal ideations and/or behaviors).

The term “therapeutically effective amount” as used herein, (for example, in describing monotherapy with intranasal esketamine) means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to therapy with a combination of agents, “therapeutically effective amount” shall mean that amount of the combination of agents taken together so that the combined effect elicits the desired biological or medicinal response. For example, the therapeutically effective amount of combination therapy comprising esketamine and a serotonin reuptake inhibitor would be the amount of esketamine and the amount of the serotonin reuptake inhibitor that when taken together or sequentially have a combined effect that is therapeutically effective, more preferably where the combined effect is synergistic. Further, it will be recognized by one skilled in the art that in the case of combination therapy with a therapeutically effect amount, the amount of each component of the combination individually may or may not be therapeutically effective.

Wherein the present invention is directed to the administration of a combination, the compounds may be co-administered simultaneously, sequentially, separately or in a single pharmaceutical composition. Where the compounds are administered separately, the number of dosages of each compound given per day, may not necessarily be the same, e.g. where one compound may have a greater duration of activity, and will therefore, be administered less frequently. Further, the compounds may be administered via the same or different routes of administration, and at the same or different times during the course of the therapy, concurrently in divided or single combination forms. The instant invention is therefore understood as embracing all regimens of simultaneous or alternating treatment and the term “administering” is to be interpreted accordingly.

As used herein, the terms “co-therapy”, “combination therapy”, “adjunctive treatment”, “adjunctive therapy” and “combined treatment” shall mean treatment of a patient in need thereof by administering esketamine in combination with one or more antidepressant(s), and further, optionally in combination with one or more atypical antipsychotics wherein the esketamine and the antidepressant(s) are administered by any suitable means, simultaneously, sequentially, separately or in a single pharmaceutical formulation. Where the esketamine and the antidepressant(s) are administered in separate dosage forms, the number of dosages administered per day for each compound may be the same or different. The esketamine and the antidepressant(s) may be administered via the same or different routes of administration. Examples of suitable methods of administration include, but are not limited to, oral, intravenous (iv), intranasal (in) intramuscular (im), subcutaneous (sc), sublingual, transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The esketamine and the antidepressant(s) may be administered according to simultaneous or alternating regimens, at the same or different times during the course of the therapy, concurrently in divided or single forms.

Optimal dosages to be administered may be readily determined by those skilled in the art, and will vary with the particular compound or compounds used, the mode of administration, the strength of the preparation and the advancement of the disease condition. In addition, factors associated with the particular patient being treated, including patient's sex, age, weight, diet, time of administration and concomitant diseases, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

As used herein, the term “composition” is intended to encompass a product comprising the specified ingredients in the specified amounts, as well as any product which results, directly or indirectly, from combinations of the specified ingredients in the specified amounts.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Val66Met rs6265
The Val66Met SNP rs6265 may be defined by SEQ ID. No. 2 (wherein R
represents the position of the polymorphism):
CTGCAGAAAG GCCTGGAATT ACAATCAGAT GGGCCACATG GCATCCCGGT GAAAGAAAGC
CC
TAACCAGTTT TCTGTCTTGT TTCTGCTTTC TCCCTACAGT TCCACCAGGT GAGAAGAGTG
ATGACCATCC TTTTCCTTAC TATGGTTATT TCATACTTTG GTTGCATGAA GGCTGCCCCC
ATGAAAGAAG CAAACATCCG AGGACAAGGT GGCTTGGCCT ACCCAGGTGT GCGGACCCAT
GGGACTCTGG AGAGCGTGAA TGGGCCCAAG GCAGGTTCAA GAGGCTTGAC ATCATTGGCT
GACACTTTCG AACAC
R
TGATAGAAGA GCTGTTGGAT GAGGACCAGA AAGTTCGGCC CAATGAAGAA AACAATAAGG
ACGCAGACTT GTACACGTCC AGGGTGATGC TCAGTAGTCA AGTGCCTTTG GAGCCTCCTC
TTCTCTTTCT GCTGGAGGAA TACAAAAATT ACCTAGATGC TGCAAACATG TCCATGAGGG
TCCGGCGCCA CTCTGACCCT GCCCGCCGAG GGGAGCTGAG CGTGTGTGAC AGTATTAGTG
AGTGGGTAAC GGCGG
CAGACAAAAA GACTGCAGTG GACATGTCGG GCGGGACGGT CACAGTCCTT GAAAAGGTCC
CTGTATCAAA AGGCCAACTG AAGCAATACT TCTACGAGAC CAAGTGCAAT CCCATGGGTT
ACACAAAAGA AGGCTGCAGG GGCATAGACA AAAGGCATTG GAACTCCCAG TGCCGAACTA
CCCAGTCGTA CGTGCGGGCC CTTACCATGG ATAGCAAAAA GAGAATTGGC TGGCGATTCA
TAAGGATAGA CACTTCTTGT GTATGTACAT TGACCATTAA AAGGGGAAGA TAGTGGATTT
ATGTTGTATA GATTAGATTA TATTGAGACA AAAATTATCT ATTTGTATAT ATACATAACA
GGGTAAATTA TTCAGTTAAG AAAAAAATAA TTTTATGAAC TGCATGTATA AATGAAGTTT
ATACAGTACA GTGGTTCTAC AATCTATTTA TTGGACATGT CCATGACCAG AAGGGAAACA
GTCATTTGCG CACAACTTAA AAAGTCTGCA TTACATTCCT TGATAATGTT GTGGTTTGTT
GCCGTTGCCA AGAACTGAAA ACATAAAAAG TTAAAAAAAA TAATAAATTG CATGCTGCTT
TAATTGTGAA TTGATAATAA ACTGTCCTCT TTCAGAAAAC AGAAAAAAAC ACACACACAC
ACAACAAAAA TTTGAACCAA AACATTCCGT TTACATTTTA GACAGTAAGT ATCTTCGTTC
TTGTTAGTAC TATATCTGTT TTACTGCTTT TAACTTCTGA TAGCGTTGGA ATTAAAACAA
TGTCAAGGTG CTGTTGTCAT TGCACCCCCA AGGGGAACTA ACCGCCTCCC ACACACTATA
TTCCTGCCAC CCCCGCCCCA CCCTACACCG GCCCCGCACC GCCCC
>gnl|dbSNP|rs6265|allelePos = 318|totalLen= 1458|taxid = 9606|
snpclass = 1|alleles = ′A/G′|mol = Genomic|build = 138

Val66Met rs6265 Genotype Testing

Genotype testing for Val66Met rs6265 polymorphism in BDNF may be completed according to known methods, for example, as described in PECIÑA M., et al., “Valence-specific effects of BDNF Val66Met polymorphism on dopaminergic stress and reward processing in humans”, J Neurosci., 2014 Apr. 23, pp 5874-81, Vol 34(17); and LIM, Y. Y, et al., (Australian Imaging, Biomarkers and Lifestyle (AIBL) Research Group), “BDNF Val66Met, Aβ amyloid, and cognitive decline in preclinical Alzheimer's disease”. Neurobiol Aging, 2013 November, pp 2457-2464, Vol. 34(11).

Additionally, genotype testing for Val66Met rs6265 polymorphism in BDNF may be accomplished using the TaqMan® SNP Genotyping Assay Kit (Catalog #4351379; rs6265, BDNF-AS, Location Chr. 11:27679916, Transition Substitution, Mis-sense, Mutation, Intragenic) available from Life Technologies™ (a brand of Thermo Fisher Scientific) (see for example http://www.lifetechnologies.com/order/genome-database/browse/genotyping/keyword/RS6265) or other commercial and experimental testing kits which can be used to perform Val66Met rs6265 genetic testing.

Val66Met rs6265 Polymorphism in BDNF and Patient Response to Ketamine and Esketamine Treatment

The effect of Val66Met rs6265 Polymorphism in BDNF Gene on Clinical response to esketamine and ketamine was investigated through a retrospective analysis of the following clinical trials, respectively: ESKETIVTRD2001 and KETIVTRD2002. Complete trial design, efficacy endpoints and pharmacogenetic testing details are available at the U.S. National Institututes of Health Clinical Trials Registry (www.clinicaltrials.gov), and are summarized in Example 1, which follows herein.

The analysis found that the Met allele of the Val66Met rs6265 polymorphism in the BDNF gene was associated with a smaller clinical response to acute, single dose ketamine or esketamine administration. In both studies, decrease in depression severity evaluated with the MADRS scale and response rates (i.e. percentage of subjects who show a response or remission based on standard clinical criteria) to the first 1-2 doses of ketamine, or esketamine were smaller in the Met carrier genotypes. The presence of at least one Met allele in the Met carrier subjects is theorized to signify a lower capacity for BDNF release, which may result into reduced synaptic plasticity; this eventually manifests clinically as lower capacity for mood improvement. Further, in the KETIVTRD2002 study, in Met carriers, the drug improvement “catches up” to the response achieved in Val homozygotes patients after repeated dosing. In the ESKETIVTRD2001 study the clinical improvement in Val homozygotes is greater than the one seen in the Met carriers after the first 1-2 administrations (day 7). In the KETIVTRD2002 study the clinical improvement seen in the Met carriers no longer differs from that of the Val homozygotes after Day 7, and at the study endpoint (Day 29), which is after repeated doses of ketamine, the depression score changes, response and remission rates are similar between the two genotype-based subgroups. Finally, there was also an effect of genotype on the durability of the clinical response following last drug dose. In ESKETIVTRD2001 the proportion of esketamine subjects who remained well in the Met carrier group was cut in half (from 6 to 3) at Day 21. In contrast all 7 of the Val/Val subjects remain well by Day 35. In KETIVTRD2002 the proportion of subjects who remain well in the Met carrier group was cut also in half (also from 7 to 4) at Day 39; while in contrast most of the Val/Val subjects (12 of 14) remain well by Day 44.

The following Examples are set forth to aid in the understanding of the invention, and are not intended and should not be construed to limit in any way the invention set forth in the claims which follow thereafter.

### Example 1

### Effect of Val66Met rs6265 Polymorphism in BDNF; Retrospective Analysis of Esketamine (ESKETIVTRD2001) and Ketamine (KETIVTRD2002) Clinical Trials

### ESKETIVTRD2001 Clinical Trial Design and Objectives:

This was a double-blind, double-randomization, placebo-controlled, multiple dose titration study in 30 adult subjects with TRD. The study consisted of 3 phases: a screening phase of up to 2 weeks, a 7-day double-blind (DB) treatment phase (Day 1 to Day 7), and a 4-week post-treatment (with optional open label [OL] esketamine 0.40 mg/kg during follow-up [FU]: administered on Days 7, 10, 14 and 17.). The interval between the first and last dose of study medication was 3 days. Approximately 30 adult subjects with TRD were randomized to treatment (esketamine 0.40 mg/kg, esketamine 0.20 mg/kg, or placebo i.v. infusion) in a 1:1:1 ratio on Day 1.

If esketamine 0.40 mg/kg dose was not well tolerated on Day 1 and/or Day 4 the dose may be reduced to 0.3 mg/kg. Subjects who were responders after the dose on Day 1 received the same treatment again on Day 4. A responder was a subject who had a reduction in MADRS total score of >50% versus baseline on Day 2, 3, or 4 (prior to dosing).

For subjects who were not responders after the dose on Day 1, the following rule was applied for treatment on Day 4:
Placebo on Day 1: re-randomization to esketamine 0.40 mg/kg or esketamine 0.20 mg/kg i.v. infusion on Day 4;
Esketamine 0.20 mg/kg on Day 1: treatment with esketamine 0.40 mg/kg i.v. infusion on Day 4;
Esketamine 0.40 mg/kg on Day 1: treatment with esketamine 0.40 mg/kg i.v. infusion again on Day 4.

One week (7 days) after the end of the double-blind treatment phase (Day 14), subjects returned to the unit for a follow-up visit. Telephone visits were conducted 3 (i.e., Day 10), 10 (i.e., Day 17), 14 (i.e., Day 21), 21 (i.e., Day 28), and 28 (i.e., Day 35) days after the end of the double-blind treatment phase. For subjects who chose it, and when agreed with the investigator, optional open label treatment of esketamine 0.40 mg/kg (or lower when required) on Days 7, 10, 14 and 17 was made available by the sponsor. The total study duration for each subject was a maximum of 7 weeks. The end of study was defined as the date of the last study assessment of the last subject in the trial.

The primary objectives of this trial were to investigate the safety and tolerability of esketamine i.v. infusion in patients with TRD; and to assess the efficacy of esketamine at 24 hours after dosing on Day 1, administered as a 0.40 mg/kg and 0.20 mg/kg intravenous (i.v.) infusion, compared with placebo in improving symptoms of depression in patients with TRD, using the Montgomery-Asberg Depression Rating Scale (MADRS).

### Clinical Endpoints:

Changes from baseline in MADRS total score after the 18 infusion (up to Day 4 pre-dose)
% responders (reduction in MADRS total score of >50% from baseline) on Days 2, 7, 17 and 35
Duration of esketamine response after the end of the OL phase (from Day 17 up to Day 35))
% remitters (MADRS total score≤10) on Days 2, 7, 17 and 35

Note: For the ESKETIVTRD2001 clinical trial, after the end of the OL phase (from Day 17 to Day 35), all responders were considered “well” (i.e. responding to the esketamine treatment). Said responders were not followed post-trial for a sufficient period of time to observe a meaningful number of relapses and therefore to determine time to relapse (empirically defined as a MADRS total score ≥22). As such, this parameter was not included in the analysis.

Additionally, although MADRS Suicidal Thoughts measurements were taken during the course of the ESKETIVTRD2001 clinical trial, subjects/patients exhibiting suicidal ideation (e.g. suicidal thoughts) at enrollment, were specifically excluded. Analysis of the effect of Val66Met polymorphism on suicidality as measured by the MADRS Suicidal Thoughts Scale was therefore deemed not applicable and not generalizable to subjects with suicidal ideation (e.g. clinically relevant)

### KETIVTRD2002 Clinical Trial Design and Objectives:

This was a double-blind, randomized, placebo-controlled, parallel arm study to assess the safety and efficacy of ketamine dosed 2 or 3 times weekly in adult subjects with treatment resistant depression. The study consisted of up to 4 phases: a screening phase of up to 4 weeks, a 4-week double-blind treatment phase (Day 1 to Day 29), an optional 2-week open label treatment phase, and a post treatment (follow up) phase of up to 3 weeks.

On Day 1 of the double-blind treatment phase, subjects were randomized to receive either i.v. infusions of placebo 2 times weekly or i.v. infusions of placebo 3 times weekly or i.v. infusions of ketamine 0.50 mg/kg, 2 times weekly or i.v. infusions of ketamine 0.50 mg/kg, 3 times weekly over 4 weeks.

Subjects who discontinued the double-blind treatment phase of the study due to lack of efficacy (based upon the clinical judgment of the investigator) after completion of the Day 15 visit, but prior to the Day 29 visit, may have received open-label ketamine treatment with the same dose frequency for an additional 2 weeks. After completing the Early Termination visit, the subject had study assessments as per the Time and Events Schedule for the optional open-label treatment phase followed by one follow up visit performed 1 week after the last dose of study medication. The total study duration for each subject was approximately 13 weeks.

The primary objectives for the trial were to assess the safety and tolerability of multiple doses over 4 weeks of ketamine administered as a 0.50 mg/kg intravenous (i.v.) infusion in subjects with TRD; and to evaluate the efficacy of ketamine administered as a 0.50 mg/kg intravenous (i.v.) infusion, 2 or 3 times weekly for 4 weeks, for the treatment of TRD compared to placebo using the Montgomery-Asberg Depression Rating Scale (MADRS) between Day 1 (pre-dose) and the (pre-dose) assessments through Day 15.

### Clinical Endpoints:

Changes in MADRS total score from baseline during the DB phase (up to Day 29)
% responders (reduction in MADRS total score of ≥50% from baseline) during the DB phase (Days 3 or 4, 8, 15, 18, 29 and End Point [DB])
Duration of ketamine response after the DB phase (from Day 29 up to Day 47
% remitters (MADRS total score≤10) on Days 3 or 4, 8, 15, and 29

Note: For the KETIVTRD2002 clinical trial, after the end of the double-blind phase (from Day 29 to Day 47), all responders were considered “well” (i.e. responding to the ketamine treatment). Said responders were not followed post-trial for a sufficient period of time to observe a meaningful number of relapses and therefore to determine time to relapse (empirically defined as a MADRS total score ≥22). As such, this parameter was not included in the analysis.

Additionally, although MADRS Suicidal Thoughts measurements were taken during the course of the KETIVTRD2002 clinical trial, subjects/patients exhibiting suicidal ideation (e.g. suicidal thoughts) at enrollment, were specifically excluded. Analysis of the effect of Val66Met polymorphism on suicidality as measured by the MADRS Suicidal Thoughts Scale was therefore deemed not applicable and not generalizable to subjects with suicidal ideation (e.g. clinically relevant).

Pooled Clinical Endpoints (Combining Results from ESKETIVTRD2001 and KETIVTRD2002)·
Changes in MADRS total score from baseline after the 1^{st} infusion (Day 2 in ESKETIVTRD2001 and Day 3 or Day 4 in KETIVTRD2002)
% responders after the 1^{st} infusion (reduction in MADRS total scores >50% on Day 2 in ESKETIVTRD2001, and reduction in MADRS total scores ≥50% on Day 3 or Day 4 in KETIVTRD2002)
% responders after multiple doses (Day 7 in ESKETIVTRD2001 and Day 8 in KETIVTRD2002)
% remitters after the 1st infusion (Day 2 in ESKETIVTRD2001 and Day 3 or Day 4 in KETIVTRD2002)

### Pharmacogenetic (DNA) Evaluation of Patients in ESKETIVTRD2001 and KETIVTRD2002 Clinical Trials:

For the ESKETIVTRD2001 study, subjects were given the option to participate in Part 1 only, Part 2 only, both parts, or neither part of the pharmacogenomic component of this study (where local regulations permit). For KETIVTRD2002 study, a 10 ml blood sample was collected from all enrolled subjects. Subject participation in Part 1 of the pharmacogenomic research was required for participation in the study. Subject participation in Part 2 was optional.

Part 1 comprised collection of pharmacogenomic samples allowed for genetic research to help understand ketamine or major depressive disorder (MDD). DNA samples were only used for genetic research related to ketamine or MDD. Genetic research consisted of the analysis of one or more candidate genes or of the analysis of genetic markers throughout the genome (as appropriate) in relation to ketamine or MDD clinical endpoints. A list of candidate genes that were potentially relevant to ketamine or MDD was provided in the study protocol. Subjects were offered the separate option to consent to storage of their optional samples for future research as scientific discoveries are made.

Part 2 of the pharmacogenomic research allowed for the storage of DNA samples for future genetic research related to ketamine or the indication(s) for which it is developed. Stored DNA samples and relevant clinical data were de-identified before research was to be done in the future. This involved removing personal identifiers and replacing the study subject identifier with a new number to limit the possibility of linking genetic data to a subject's identity.

The genetic endpoint was determination of a patient's Brain-Derived Neurotrophic Factor (BDNF) gene/polymorphism SNP (rs6265). Allele and genotype frequencies (counts) were calculated for all subjects from pooled

ESKETIVTRD2001 and KETIVTRD2002 studies. Observed minor allele A (or T) frequency and genotyping frequency were compared to the reported frequency (in European population, frequency of minor allele A (or T) is ˜20%; frequencies for A/A (or T/T), A/G (or T/C) and Val/Val G/G (or C/C) are ˜3%, ˜34%, and ˜64%, respectively).

### Efficacy Endpoint Analysis:

Summary statistics for changes in MADRS total score by Val/Val or Met carriers and by treatment regimen were determined for each study separately (up to Day 4 pre-dose in ESKETIVTRD2001, and up to Day 29 in KETIVTRD2002) and as well as for two studies pooled (after the 1^{st} infusion: Day 2 in ESKETIVTRD2001 and Day 3 or Day 4 in KETIVTRD2002). Mean plots in changes in MADRS total score over time by Val/Val or Met carriers and by treatment regimen were determined for each study separately (up to Day 4 pre-dose in ESKETIVTRD2001 and up to Day 15 and Day 29 in KETIVTRD2002). Individual MADRS total score and as well as changes in MADRS total score were determined for each study separately (after the open label phase, Day 17 in ESKETIVTRD2001 and after the double blind phase Day 29 in KETIVTRD2002).

Proportions of responders or remitters at various time points by Val/Val or Met carriers and by treatment regimen (per Day 1 randomization for EDKETIVTRD2001) were calculated for each study separately (Days 2, 7, 17 and 35 in EDKETIVTRD2001 and Days 3 or 4, 8, 15, and 29 in KETIVTRD2002). Likewise, proportions of responders or remitters by Val/Val or Met carriers in all subjects (pooled placebo and esketamine) in EDKETIVTRD2001 were calculated. In addition, response rates at Day 18 (FU) and End Point (DB) in KETIVTRD2002 were calculated.

The association between changes in MADRS total score from baseline (Day 2 in TESKETIVRD2001 and Day 3 or Day 4 in KETIVTRD2002) and the SNP (rs6265) were evaluated in placebo (pooled from ESKETIVTRD2001 and KETIVTRD2002) and active (pooled esketamine from ESKETIVTRD2001 and ketamine KETIVTRD2002) groups using an ANCOVA model under a dominant model with the baseline MARDS total score and genotype.

The association between the % responders or the % remitters (Day 2 in ESKETIVTRD2001, and Day 3 or Day 4 in KETIVTRD2002) and the SNP (rs6265) were evaluated in placebo (pooled from ESKETIVTRD2001 and KETIVTRD2002) and active (pooled esketamine from ESKETIVTRD2001 and ketamine KETIVTRD2002) groups using a logistic regression model under a dominant model with the baseline MADRS total score and genotype.

The duration of response (after the open label phase Day 17 in

ESKETIVTRD2001, and after the double blind phase Day 29 in KETIVTRD2002) was evaluated using Kaplan-Meier method with the strata of carrier (Val/Val or Met carriers). The survival curves of the duration of response by Val/Val or Met carriers were plotted for each study separately.

Time to relapse in all responders (after the open label phase Day 17 in EDKETIVTRD2001 and after the double blind phase Day 29 in KETIVTRD2002) was evaluated using Kaplan-Meier method with the strata of carrier (Val/Val or Met carriers). The survival curves of time to relapse by Val/Val or Met carriers were plotted for each study separately.

### Analysis Results

The analysis set contained 93 subjects, including 41 in placebo, 17 in ESKETIVTRD2001 and 35 in KETIVTRD2002. Hardy-Weinberg Equillibrium (HWE) analysis found that genotype frequency observed in the two clinical trials was in line with the reported frequency in European populations. Analysis was completed for patients in the ESKETIVTRD2001 and KETIVTRD2002 clinical trials individually, and pooled, with results as detailed below.

Results from Pooled ESKETIVTRD2001 and KETIVTRD2002 Clinical Trials Analysis

A) MADRS Scores: Mean changes in MADRS total score from baseline after the 1^{st} infusion (Day 2 in TRD2001 and Day 3 or Day 4 in TRD2002) by Val/Val or Met carriers were as listed in Table P-1, below. Larger reductions were observed in Val/Val subjects as compared to Met carriers for both placebo and active (esketamine or ketamine).

TABLE P-1
MADRS Total Scores, Actual Values and Changes from Baseline After
the First Infusion (Day 2 in TRD2001 and Day 3 or Day 4 TRD2002)
by Val/Val or Met Carriers and by Active or Placebo

Change from Baseline

| | | | |
|---|---|---|---|
| No. | Mean (SD) | No. | Mean (SD) |

Val/Val

| | | | | | | |
|---|---|---|---|---|---|---|
| Placebo | 29 | 30.48 | (8.100) | 29 | −5.62 | (7.218) |
| Active | 29 | 18.93 | (8.980) | 29 | −15.14 | (9.054) |

Met Carriers

| | | | | | | |
|---|---|---|---|---|---|---|
| Placebo | 11 | 33.27 | (5.850) | 11 | −1.64 | (5.697) |
| Active | 22 | 24.27 | (8.072) | 22 | −8.86 | (7.511) |

Placebo: pooled from placebos from esketamine and ketamine
Active: pooled from low, high dose from esketamine and 2X/WK, 3X/WK from ketamine

B) % Responders: The % responders after the 1^{st} infusion (Day 2 in TRD2001 and Day 3 or Day 4 in TRD2002) by Val/Val or Met carriers and by placebo or active, and by esketamine or ketamine were as listed in Table P-2 below. Response rates were higher in Val/Val subjects than in Met carriers.

TABLE P-2
MADRS Total Scores - Proportion of Responders After
the First Infusion (Day 2 in TRD2001 and Day 3 or Day 4
in TRD2002) by Val/Val or Met Carriers and by Esketamine,
Ketamine, Pooled Active or Placebo in All Subjects
Pooled Subjects

| | | |
|---|---|---|
| Responders N (%) | Non-responders N (%) | Total |

Val/Val

| | | | | | |
|---|---|---|---|---|---|
| Placebo | 2 | (6.9%) | 27 | (93.1%) | 29 |
| Active | 12 | (41.4%) | 17 | (58.6%) | 29 |

Met Carriers

| | | | | | |
|---|---|---|---|---|---|
| Placebo | 0 | (0%) | 11 | (100.0%) | 11 |
| Active | 2 | (9.1%) | 20 | (90.9%) | 22 |

Val/Val

| | | | | | |
|---|---|---|---|---|---|
| Placebo | 2 | (6.9%) | 27 | (93.1%) | 29 |
| Esketamine | 6 | (75.0%) | 2 | (25.0%) | 8 |
| Ketamine | 6 | (28.6%) | 15 | (71.4%) | 21 |

Met Carriers

| | | | | | |
|---|---|---|---|---|---|
| Placebo | 0 | (0%) | 11 | (100.0%) | 11 |
| Esketamine | 1 | (11.1%) | 8 | (88.9%) | 9 |
| Ketamine | 1 | (7.7%) | 12 | (92.3%) | 13 |

Placebo: pooled from placebos from Esketamine and Ketamine
Active: pooled from low, high dose from Esketamine and 2X/WK, 3X/WK from Ketamine
Placebo: pooled from placebos from Esketamine and Ketamine
Esketamine: pooled from low, high dose
Ketamine: pooled from 2X/WK, 3X/WK from JNJ-644059

C) % Remitters: The % remitters after the 1^{st} infusion (Day 2 in TRD2001 and Day 3 or Day 4 in TRD2002) by Val/Val or Met carriers and by placebo or active were as listed in Table P-3, below. Remit rate was higher in Val/Val subjects than in Met carriers.

TABLE P-3
MADRS Total Scores - Proportion of Remitters After the First Infusion
(Day 2 in TRD2001 and Day 3 or Day 4 in TRD2002) by Val/Val or
Met Carriers, and by Active or Placebo in All Subjects

| | | |
|---|---|---|
| Remitters No. (%) | Non-remitters No. (%) | Total |

Val/Val

| | | | | | |
|---|---|---|---|---|---|
| Placebo | 1 | (3.4%) | 28 | (96.6%) | 29 |
| Active | 6 | (20.7%) | 23 | (79.3%) | 29 |

Met Carriers

| | | | | | |
|---|---|---|---|---|---|
| Placebo | 0 | (0%) | 11 | (100.0%) | 11 |
| Active | 1 | (4.5%) | 21 | (95.5%) | 22 |

Placebo: pooled from placebos from Esketamine and Ketamine
Active: pooled from low, high dose from Esketamine and 2X/WK, 3X/WK from Ketamine

### Association Analysis (Summary Results):

Association analysis between efficacy endpoints and SNP rs6265 were performed in placebo subjects (pooled from ESKETIVTRD2001 and KETIVTRD2002) and as well as in active subjects (pooled esketamine subjects from ESKETIVTRD2001 or ketamine subjects from KETIVTRD2002). Changes in MADRS total score from baseline after a single dose were based on Day 2 (for ESKETIVTRD2001) and Day 3 or Day 4 (for KETIVTRD2002).

Association between changes in MADRS total score from baseline and the SNP rs6265 after a single dose was evaluated in placebo subjects and active subjects using an ANCOVA model under a dominant model with baseline MADRS total score, and grouped genotype (Val/Val or Met carrier). A statistically significant result was found in active subjects (p=0.02). However, there was no evidence of association between change in MADRS total score and the SNP rs6265 in placebo subjects (p=0.14).

Association between the % responders and the SNP rs6265 after a single dose was evaluated in placebo subjects and active subjects using a logistic regression model under a dominant model with baseline MADRS total score and grouped genotype (Val/Val or Met carrier). A statistically significant result was found in active subjects (p=0.03). However, there was no evidence of association between % of responders and the SNP rs6265 in placebo subjects (p=0.68).

Association between the % remitters and the SNP rs6265 after a single dose was evaluated in placebo subjects and as well as in active subjects using a logistic regression model under a dominant model with baseline MADRS total score and grouped genotype (Val/Val or Met carrier). There was no evidence of association between the % remitters and the SNP rs6265 in placebo subjects or active subjects.

### ESKETIVTRD2001 Analysis Results

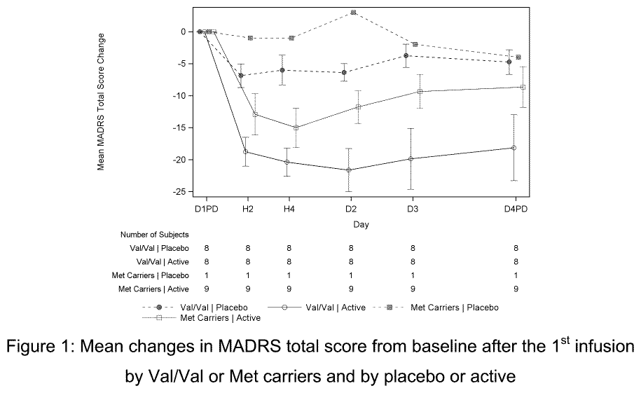

A) MADRS Scores: Larger reductions in MADRS total score were observed in Val/Val subjects as compared to Met carriers for both placebo and esketamine. Mean changes in MADRS total score from baseline after the 1^{st} infusion (up to Day 4) by Val/Val or Met carriers and by placebo or active (pooled esketamine 0.2 mg/kg and 0.4 mg/kg, per Day 1 randomization) are as plotted in FIG. 1 and listed in Table E-1.

TABLE E-1
MADRS Score
Mean Change from Baseline up to Day 4 pre-dose

| | | | | | | |
|---|---|---|---|---|---|---|
| | | | Base | | | |
| N | Mean (SD) | Range | Mean | N | Mean (SD) | Range |

Val/Val Subjects

| | | | | | | | |
|---|---|---|---|---|---|---|---|
| Placebo | | | | | | | |
| D1PD | 8 | 34.63 (4.340) | (30.0; 42.0) | 34.63 | | | |
| D1H2 | 8 | 27.75 (4.132) | (21.0; 32.0) | 34.63 | 8 | −6.88 (5.194) | (−15.0; 0.0) |
| D1H4 | 8 | 28.63 (5.805) | (18.0; 37.0) | 34.63 | 8 | −6.00 (6.568) | (−16.0; 2.0) |
| D2 | 8 | 28.25 (4.097) | (22.0; 36.0) | 34.63 | 8 | −6.38 (3.889) | (−12.0; 1.0) |
| D3 | 8 | 30.88 (4.853) | (24.0; 36.0) | 34.63 | 8 | −3.75 (5.148) | (−11.0; 4.0) |
| D4PD | 8 | 29.88 (6.105) | (18.0; 38.0) | 34.63 | 8 | −4.75 (5.445) | (−13.0; 0.0) |
| Active | | | | | | | |
| D1PD | 8 | 31.75 (5.007) | (23.0; 41.0) | 31.75 | | | |
| D1H2 | 8 | 13.00 (5.904) | (2.0; 18.0) | 31.75 | 8 | −18.75 (6.386) | (−29.0; −9.0) |
| D1H4 | 8 | 11.38 (4.926) | (2.0; 17.0) | 31.75 | 8 | −20.38 (6.186) | (−29.0; −12.0) |
| D2 | 8 | 10.13 (6.105) | (0.0; 19.0) | 31.75 | 8 | −21.63 (9.501) | (−33.0; −6.0) |
| D3 | 8 | 11.88 (10.494) | (0.0; 29.0) | 31.75 | 8 | −19.88 (13.474) | (−38.0; −3.0) |
| D4PD | 8 | 13.63 (11.698) | (1.0; 30.0) | 31.75 | 8 | −18.13 (14.515) | (−37.0; 0.0) |

Met Carriers

| | | | | | | | |
|---|---|---|---|---|---|---|---|
| Placebo | | | | | | | |
| D1PD | 1 | 30 (—) | (30.0; 30.0) | 30.00 | | | |
| D1H2 | 1 | 29 (—) | (29.0; 29.0) | 30.00 | 1 | −1 (—) | (−1.0; −1.0) |
| D1H4 | 1 | 29 (—) | (29.0; 29.0) | 30.00 | 1 | −1 (—) | (−1.0; −1.0) |
| D2 | 1 | 33 (—) | (33.0; 33.0) | 30.00 | 1 | 3 (—) | (3.0; 3.0) |
| D3 | 1 | 28 (—) | (28.0; 28.0) | 30.00 | 1 | −2 (—) | (−2.0; −2.0) |
| D4PD | 1 | 26 (—) | (26.0; 26.0) | 30.00 | 1 | −4 (—) | (−4.0; −4.0) |
| Active | | | | | | | |
| D1PD | 9 | 33.67 (3.937) | (28.0; 42.0) | 33.67 | | | |
| D1H2 | 9 | 20.78 (9.985) | (4.0; 34.0) | 33.67 | 9 | −12.89 (9.765) | (−26.0; 0.0) |
| D1H4 | 9 | 18.67 (10.198) | (6.0; 34.0) | 33.67 | 9 | −15.00 (9.287) | (−28.0; −2.0) |
| D2 | 9 | 21.89 (8.580) | (4.0; 33.0) | 33.67 | 9 | −11.78 (7.645) | (−30.0; −3.0) |
| D3 | 9 | 24.33 (9.028) | (9.0; 33.0) | 33.67 | 9 | −9.33 (8.000) | (−23.0; 0.0) |
| D4PD | 9 | 25.00 (10.452) | (5.0; 36.0) | 33.67 | 9 | −8.67 (9.566) | (−29.0; 0.0) |

B) % Responders: The % responders by Val/Val or Met carriers and by placebo or esketamine (per Day 1 randomization, however, all patients got active esketamine from day 4 on) on Days 2, 7, 17 and 35 were as listed in Table E-2, below. In esketamine subjects, response rates were higher in Val/Val subjects than in Met carriers

TABLE E-2
MADRS Total Scores, Proportion of Responders on Days
2, 7, 17 and 35 by Val/Val or Met Carriers and by
Esketamine or Placebo (per Day 1 Randomization)

| | | |
|---|---|---|
| Responders No. (%) | Non-responders No. (%) | Total |

Val/Val

| Placebo | | | | | |
|---|---|---|---|---|---|
| DAY 02 | 0 | | 8 | (100.0%) | 8 |
| DAY 07* | 5 | (71.4%) | 2 | (28.6%) | 7 |
| DAY 17* | 5 | (71.4%) | 2 | (28.6%) | 7 |
| DAY 35* | 4 | (57.1%) | 3 | (42.9%) | 7 |
| Esketamine | | | | | |
| DAY 02 | 6 | (75.0%) | 2 | (25.0%) | 8 |
| DAY 07 | 6 | (75.0%) | 2 | (25.0%) | 8 |
| DAY 17 | 7 | (87.5%) | 1 | (12.5%) | 8 |
| DAY 35 | 7 | (87.5%) | 1 | (12.5%) | 8 |

Met Carriers

| Placebo | | | | | |
|---|---|---|---|---|---|
| DAY 02 | 0 | | 1 | (100.0%) | 1 |
| DAY 07* | 1 | (100.0%) | 0 | | 1 |
| DAY 17* | 1 | (100.0%) | 0 | | 1 |
| DAY 35* | 1 | (100.0%) | 0 | | 1 |
| Esketamine | | | | | |
| DAY 02 | 1 | (11.1%) | 8 | (88.9%) | 9 |
| DAY 07 | 3 | (33.3%) | 6 | (66.7%) | 9 |
| DAY 17 | 6 | (66.7%) | 3 | (33.3%) | 9 |
| DAY 35 | 4 | (44.4%) | 5 | (55.6%) | 9 |

*All patients listed as “placebo” received active esketamine from day 4 onward. Therefore the response rates reported here from day 7 onwards as placebo (per day 1 randomization) reflect response to esketamine.

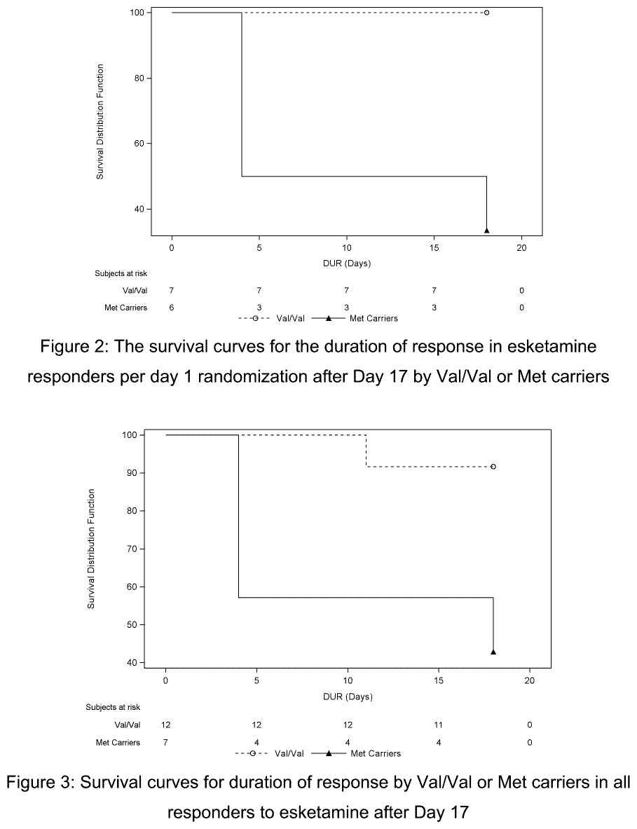

C) Duration of Response: The survival curves for the duration of response in esketamine responders per day 1 randomization after the open label active treatment phase (Day 17) by Val/Val or Met carriers in esketamine responders (Day 17) were as shown in FIG. 2 and Table E-3, below. In esketamine subjects (per Day 1 randomization), there were 13 responders on Day 17 (7 with Val/Val and 6 with Met carriers). Among those responders, all 7 (100%) Val/Val subjects maintained response status up to Day 35, i.e. 18 days after last dose. For the Met carriers, 2 out of 6 (33.3%) maintained response status up to Day 35.

TABLE E-3
Survival Function Estimates on the Duration of Response
After Open Label Phase by Val/Val or Met Carriers in
Esketamine Responders (Day 17) Per Day 1 Randomization

| | | | | | |
|---|---|---|---|---|---|
| Day | Survival | Failure | Survival Std. Err. | No. Failed | No. Left |

Val/Val Subjects

| | | | | | |
|---|---|---|---|---|---|
| 0S̊ | 1.0000 | 0 | 0 | 0 | 7 |
| 18* | | | | 0 | 6 |
| 18* | | | | 0 | 5 |
| 18* | | | | 0 | 4 |
| 18* | | | | 0 | 3 |
| 18* | | | | 0 | 2 |
| 18* | | | | 0 | 1 |
| 18* | | | | 0 | 0 |

Met Carriers

| | | | | | |
|---|---|---|---|---|---|
| 0S̊ | 1.0000 | 0 | 0 | 0 | 6 |
| 4S̊ | | | | 1 | 5 |
| 4S̊ | | | | 2 | 4 |
| 4S̊ | 0.5000 | 0.5000 | 0.2041 | 3 | 3 |
| 18S̊ | 0.3333 | 0.6667 | 0.1925 | 4 | 2 |
| 18* | | | | 4 | 1 |
| 18* | | | | 4 | 0 |

*sensored observations

The survival curves for the aforementioned duration of response by Val/Val or Met carriers in all responders to ketamine (Day 17) were as shown in FIG. 3 (FIG. 3B) and Table E-4, below. Among all subjects, there were 19 responders on Day 17 (12 with Val/Val and 7 with Met carriers). Among those responders, 11 out of 12 (91.7%) Val/Val maintained response status up to Day 35. For the Met carriers, 3 out of 7 (42.9%) maintained response status up to Day 35.

TABLE E-4
Survival Function Estimates on the Duration of Response
on Day 17 by Val/Val or Met Carriers in All Responders

| | | | | | |
|---|---|---|---|---|---|
| Day | Survival | Failure | Survival Std. Err. | No. Failed | No. Left |

Val/Val Subjects

| | | | | | |
|---|---|---|---|---|---|
| 0S̊ | 1.0000 | 0 | 0 | 0 | 12 |
| 11S̊ | 0.9167 | 0.0833 | 0.0798 | 1 | 11 |
| 18* | | | | 1 | 10 |
| 18* | | | | 1 | 9 |
| 18* | | | | 1 | 8 |
| 18* | | | | 1 | 7 |
| 18* | | | | 1 | 6 |
| 18* | | | | 1 | 5 |
| 18* | | | | 1 | 4 |
| 18* | | | | 1 | 3 |
| 18* | | | | 1 | 2 |
| 18* | | | | 1 | 1 |
| 18* | | | | 1 | 0 |

Met Carriers

| | | | | | |
|---|---|---|---|---|---|
| 0S̊ | 1.0000 | 0 | 0 | 0 | 7 |
| 4S̊ | | | | 1 | 6 |
| 4S̊ | | | | 2 | 5 |
| 4S̊ | 0.5714 | 0.4286 | 0.1870 | 3 | 4 |
| 18S̊ | 0.4286 | 0.5714 | 0.1870 | 4 | 3 |
| 18* | | | | 4 | 2 |
| 18* | | | | 4 | 1 |
| 18* | | | | 4 | 0 |

*sensored observations

D) % Remitters: The % remitters by Val/Val or Met carriers and by placebo or esketamine (per Day 1 randomization) on Days 2, 7, 17 and 35 were as listed in table E-5 below. In esketamine subjects, remit rates were higher in Val/Val subjects than in Met carriers at all timepoints. Remission was maintained longer in Val/Val than in Met carriers (Day 35).

TABLE E-5
MADRS Total cores, Proportion of Remitters on Days 2, 7, 17 and
35 by Val/Val or Met Carriers and by Esketamine or Placebo

| | | |
|---|---|---|
| Remitters No. (%) | Non-remitters No. (%) | Total |

Val/Val

| Placebo | | | | | |
|---|---|---|---|---|---|
| DAY 02 | 0 | | 8 | (100.0%) | 8 |
| DAY 07* | 4 | (57.1%) | 3 | (42.9%) | 7 |
| DAY 17* | 3 | (42.9%) | 4 | (57.1%) | 7 |
| DAY 35* | 4 | (57.1%) | 3 | (42.9%) | 7 |
| Esketamine | | | | | |
| DAY 02 | 5 | (62.5%) | 3 | (37.5%) | 8 |
| DAY 07 | 6 | (75.0%) | 2 | (25.0%) | 8 |
| DAY 17 | 7 | (87.5%) | 1 | (12.5%) | 8 |
| DAY 35 | 7 | (87.5%) | 1 | (12.5%) | 8 |

Met Carriers

| Placebo | | | | | |
|---|---|---|---|---|---|
| DAY 02 | 0 | | 1 | (100.0%) | 1 |
| DAY 07* | 0 | | 1 | (100.0%) | 1 |
| DAY 17* | 1 | (100.0%) | 0 | | 1 |
| DAY 35* | 1 | (100.0%) | 0 | | 1 |
| Esketamine | | | | | |
| DAY 02 | 1 | (11.1%) | 8 | (88.9%) | 9 |
| DAY 07 | 2 | (22.2%) | 7 | (77.8%) | 9 |
| DAY 17 | 2 | (22.2%) | 7 | (77.8%) | 9 |
| DAY 35 | 2 | (22.2%) | 7 | (77.8%) | 9 |

*All patients listed as “placebo” received active esketamine from day 4 onward. Therefore the remission rates reported here from day 7 onwards as placebo (per day 1 randomization) reflect remission to esketamine

### KETIVTRD2002 Analysis Results

A) MADRS Scores: Larger reductions in MADRS total score over most time points were observed in Val/Val carriers as compared to those Met carriers for both placebo and ketamine (pooled 2×/wk and 3×/wk). Summary statistics for MADRS total score over time by Val/Val or Met carriers and by ketamine 2×/wk, ketamine 3×/wk or placebo were as listed in Table K-1, below.

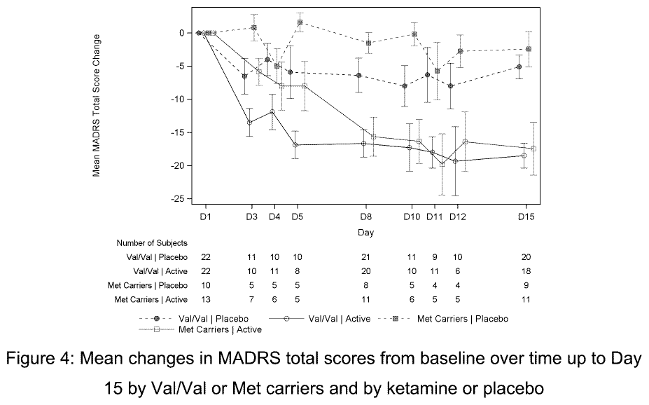

Similarly, mean changes in MADRS total scores from baseline over time (during the double-blinded phase) up to the primary endpoint at Day 15 by Val/Val or Met carriers and by ketamine or placebo were shown in FIG. 4. (Note: The study continued beyond Day 15, however results from Day 15 onward are not presented since at Day 15, non-responders were permitted to received open label Ketamine, as per the clinical trial desgin.) Summary statistics for mean changes in MADRS total scores from baseline by Val/Val or Met carriers and by ketamine (pooled 2×/wk and 3×/wk) or placebo were as listed in Table K-1, below.

TABLE K-1
MADRS Total Scores - Actual Values and Changes from Baseline on Days 3 or
4, 8, 15 and 29 by Val/Val or Met Carriers and by Ketamine or Placebo

| | | | | Change from Baseline | | | |
|---|---|---|---|---|---|---|---|
| | Mean | | Base | | Mean | Std. | |
| N | (SD) | Range | Mean | N | (SD) | Err. | Range |

Val/Val Placebo

| | | | | | | | | |
|---|---|---|---|---|---|---|---|---|
| DAY 03 or | 21 | 31.33 | (9.0; 43.0) | 36.67 | 21 | −5.33 | 1.791 | (−31.0; 2.0) |
| DAY 04 | | (9.123) | | | | (8.206) | | |
| DAY 08(DB) | 21 | 30.29 | (3.0; 48.0) | 36.67 | 21 | −6.38 | 2.588 | (−37.0; 6.0) |
| | | (13.150) | | | | (11.859) | | |
| DAY 15(DB) | 20 | 31.25 | (10.0; 48.0) | 36.35 | 20 | −5.10 | 1.802 | (−26.0; 4.0) |
| | | (9.673) | | | | (8.058) | | |
| DAY 29(DB) | 2 | 11.50 | (5.0; 18.0) | 35.00 | 2 | −23.50 | 7.500 | (−31.0; −16.0) |
| | | (9.192) | | | | (10.607) | | |

Val/Val Ketamine

| | | | | | | | | |
|---|---|---|---|---|---|---|---|---|
| DAY 03 or | 21 | 22.29 | (6.0; 34.0) | 34.95 | 21 | −12.67 | 1.689 | (−27.0; 0.0) |
| DAY 04 | | (7.551) | | | | (7.742) | | |
| DAY 08(DB) | 20 | 18.50 | (6.0; 34.0) | 35.15 | 20 | −16.65 | 2.082 | (−38.0; −4.0) |
| | | (8.030) | | | | (9.309) | | |
| DAY 15(DB) | 18 | 16.11 | (5.0; 34.0) | 34.61 | 18 | −18.50 | 1.863 | (−34.0; −4.0) |
| | | (8.159) | | | | (7.906) | | |
| DAY 29(DB) | 17 | 9.59 | (0.0; 26.0) | 36.18 | 17 | −26.59 | 2.168 | (−43.0; −11.0) |
| | | (7.969) | | | | (8.938) | | |

Met Carriers Placebo

| | | | | | | | | |
|---|---|---|---|---|---|---|---|---|
| DAY 03 or | 10 | 33.30 | (24.0; 40.0) | 35.40 | 10 | −2.10 | 1.828 | (−15.0; 8.0) |
| DAY 04 | | (6.165) | | | | (5.782) | | |
| DAY 08(DB) | 8 | 33.63 | (29.0; 41.0) | 35.13 | 8 | −1.50 | 1.581 | (−10.0; 5.0) |
| | | (4.406) | | | | (4.472) | | |
| DAY 15(DB) | 9 | 33.11 | (17.0; 40.0) | 35.56 | 9 | −2.44 | 2.652 | (−22.0; 5.0) |
| | | (6.882) | | | | (7.955) | | |
| DAY 29(DB) | 1 | 31 | (31.0; 31.0) | 32.00 | 1 | −1 | | (−1.0; −1.0) |
| | | (—) | | | | (—) | | |

Met Carriers Ketamine

| | | | | | | | | |
|---|---|---|---|---|---|---|---|---|
| DAY 03 or | 13 | 25.92 | (14.0; 38.0) | 32.77 | 13 | −6.85 | 1.941 | (−22.0; 4.0) |
| DAY 04 | | (7.599) | | | | (6.998) | | |
| DAY 08(DB) | 11 | 17.00 | (4.0; 33.0) | 32.64 | 11 | −15.64 | 2.940 | (−33.0; −4.0) |
| | | (9.930) | | | | (9.750) | | |
| DAY 15(DB) | 11 | 15.18 | (1.0; 44.0) | 32.64 | 11 | −17.45 | 3.969 | (−36.0; 10.0) |
| | | (13.060) | | | | (13.163) | | |
| DAY 29(DB) | 9 | 10.78 | (2.0; 32.0) | 32.78 | 9 | −22.00 | 2.848 | (−33.0; −5.0) |
| | | (8.955) | | | | (8.544) | | |

B) % Responders: Among ketamine subjects, response rates were higher on Day 3 or Day 4 and Day 15 in Val/Val subjects than in Met carriers, whereas response rates were similar on Days 8 and 29 in Val/Val subjects than in Met carriers. The % responders during the double blind phase by Val/Val or Met 5 carriers and by ketamine (pooled 2×/wk and 3×/wk) or placebo were as listed in Table K-2 below.

TABLE K-2
MADRS Total Scores - Proportion of Responders on Days 3 or 4,
8, 15 and 29 by Val/Val or Met Carriers and by Treatment Group

| | | |
|---|---|---|
| Responders N (%) | Non-responders N (%) | Total |

Val/Val

| | | | | | |
|---|---|---|---|---|---|
| Placebo | | | | | |
| DAY 03 or DAY 04 | 2 | (9.5%) | 19 | (90.5%) | 21 |
| DAY 08(DB) | 4 | (19.0%) | 17 | (81.0%) | 21 |
| DAY 15(DB) | 2 | (10.0%) | 18 | (90.0%) | 20 |
| DAY 29(DB) | 1 | (50.0%) | 1 | (50.0%) | 2 |
| Ketamine Pooled | | | | | |
| DAY 03 or DAY 04 | 6 | (28.6%) | 15 | (71.4%) | 21 |
| DAY 08(DB) | 8 | (40.0%) | 12 | (60.0%) | 20 |
| DAY 15(DB) | 12 | (66.7%) | 6 | (33.3%) | 18 |
| DAY 29(DB) | 14 | (82.4%) | 3 | (17.6%) | 17 |

Met Carriers

| Placebo | | | | | |
|---|---|---|---|---|---|
| DAY 03 or DAY 04 | 0 | | 10 | (100.0%) | 10 |
| DAY 08(DB) | 0 | | 8 | (100.0%) | 8 |
| DAY 15(DB) | 1 | (11.1%) | 8 | (88.9%) | 9 |
| DAY 29(DB) | 0 | | 1 | (100.0%) | 1 |
| Ketamine Pooled | | | | | |
| DAY 03 or DAY 04 | 1 | (7.7%) | 12 | (92.3%) | 13 |
| DAY 08(DB) | 5 | (45.5%) | 6 | (54.5%) | 11 |
| DAY 15(DB) | 6 | (54.5%) | 5 | (45.5%) | 11 |
| DAY 29(DB) | 8 | (88.9%) | 1 | (11.1%) | 9 |

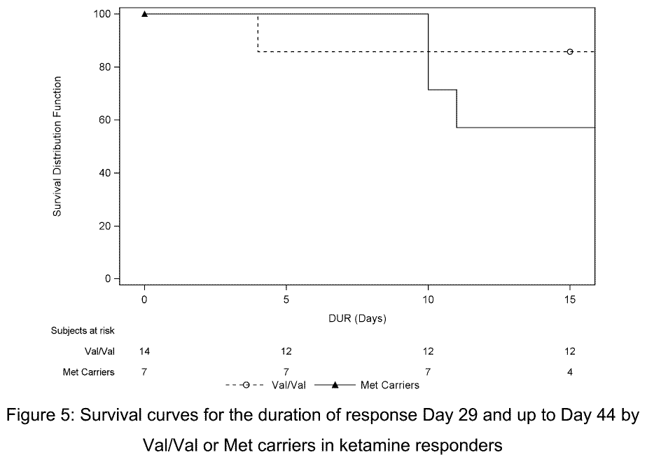

C) Duration of Response: The survival curves for the duration of response after the double-blind phase Day 29 and up to Day 44 (i.e., up to 15 days after the last dose) by Val/Val or Met carriers in ketamine responders on Day 29 were as shown in FIG. 5 (where on the x-axis, 0 represents the starting point at Day 29 and 15 represents Day 44) and as listed in Table K-3, below. There were 22 ketamine responders on Day 29 (14 with Val/Val and 8 with Met carriers). Among those responders, 9 out of 14 (64.3%) in Val/Val maintain response status, versus 5 out of 8 (62.5%) in Met carriers maintain response status during the follow-up phase.

TABLE K-3
Survival Function Estimates on the Duration of
Response During Follow-up Phase by Val/Val or
Met Carriers in Ketamine Responders (Day 29)

| | | | | | |
|---|---|---|---|---|---|
| | | | Survival Std. | | |
| Day | Survival | Failure | Err. | No. Failed | No. Left |

Val/Val Subjects

| | | | | | |
|---|---|---|---|---|---|
| 0S̊ | 1.0000 | 0 | 0 | 0 | 14 |
| 4S̊ | | | | 1 | 13 |
| 4S̊ | 0.8571 | 0.1429 | 0.0935 | 2 | 12 |
| 15S̊ | | | | 2 | 11 |
| 16S̊ | 0.7792 | 0.2208 | 0.1129 | 3 | 10 |
| 17* | | | | 3 | 9 |
| 17* | | | | 3 | 8 |
| 17* | | | | 3 | 7 |
| 18S̊ | 0.6679 | 0.3321 | 0.1414 | 4 | 6 |
| 18* | | | | 4 | 5 |
| 18* | | | | 4 | 4 |
| 18* | | | | 4 | 3 |
| 18* | | | | 4 | 2 |
| 22* | | | | 4 | 1 |
| 38S̊ | 0 | 1.0000 | | 5 | 0 |

Met Carriers

| | | | | | |
|---|---|---|---|---|---|
| 0S̊ | 1.0000 | 0 | 0 | 0 | 8 |
| 0* | | | | 0 | 7 |
| 10S̊ | | | | 1 | 6 |
| 10S̊ | 0.7143 | 0.2857 | 0.1707 | 2 | 5 |
| 11S̊ | 0.5714 | 0.4286 | 0.1870 | 3 | 4 |
| 17* | | | | 3 | 3 |
| 17* | | | | 3 | 2 |
| 18* | | | | 3 | 1 |
| 30* | | | | 3 | 0 |

*sensored observations

D) % Remitters: Among ketamine subjects, remit rates were higher in Met carriers than in Val/Val subjects, except on Day 3 or Day 4. Among placebo subjects, there were no remitters in Met carriers. The results for % remitters by Val/Val or Met carriers and by ketamine (pooled 2×/week and 3×/week) or placebo were as presented in Table K-4.

TABLE K-4
MADRS Total Scores - Proportion of Remitters
on Days 3 or 4, 8, 15 and 29 by Val/Val or
Met Carriers and by Treatment Group No. (%)

| | | |
|---|---|---|
| Remitters No. (%) | Non-remitters No. (%) | Total |

Val/Val

| | | | | | |
|---|---|---|---|---|---|
| Placebo | | | | | |
| DAY 03 or DAY 04 | 1 | (4.8%) | 20 | (95.2%) | 21 |
| DAY 08(DB) | 3 | (14.3%) | 18 | (85.7%) | 21 |
| DAY 15(DB) | 1 | (5.0%) | 19 | (95.0%) | 20 |
| DAY 29(DB) | 1 | (50.0%) | 1 | (50.0%) | 2 |
| Ketamine Pooled | | | | | |
| DAY 03 or DAY 04 | 1 | (4.8%) | 20 | (95.2%) | 21 |
| DAY 08(DB) | 4 | (20.0%) | 16 | (80.0%) | 20 |
| DAY 15(DB) | 4 | (22.2%) | 14 | (77.8%) | 18 |
| DAY 29(DB) | 11 | (64.7%) | 6 | (35.3%) | 17 |

Met Carriers

| Placebo | | | | | |
|---|---|---|---|---|---|
| DAY 03 or DAY 04 | 0 | | 10 | (100.0%) | 10 |
| DAY 08(DB) | 0 | | 8 | (100.0%) | 8 |
| DAY 15(DB) | 0 | | 9 | (100.0%) | 9 |
| DAY 29(DB) | 0 | | 1 | (100.0%) | 1 |
| Ketamine Pooled | | | | | |
| DAY 03 or DAY 04 | 0 | | 13 | (100.0%) | 13 |
| DAY 08(DB) | 3 | (27.3%) | 8 | (72.7%) | 11 |
| DAY 15(DB) | 5 | (45.5%) | 6 | (54.5%) | 11 |
| DAY 29(DB) | 6 | (66.7%) | 3 | (33.3%) | 9 |

### Formulation Example 1—Prophetic Example

An aqueous formulation of S-ketamine hydrochloride is prepared by mixing S-ketamine hydrochloride (at a concentration of 161.4 mg/ml) in water and then adding 1N NaOH_{(aq)} to pH 5.0.

### Formulation Example 2—Prophetic Example

Aqueous formulation of S-ketamine hydrochloride is prepared by mixing S-ketamine hydrochloride (at a concentration of 161.4 mg/mL) in water and then adding 10 mg/ml tauroursodeoxycholic acid (TUDCA). To the resulting mixture is added 1N NaOH_{(aq)} to pH 4.5.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

BRIEF DESCRIPTION OF SEQUENCE LISTINGS

| SEQ | Sequence |
|---|---|
| ID | Val66Met polymorphysm on BDNF, |
| NO. | dbSNP rs6265 [Homo sapien] |
| 1 | ATCATTGGCTGACACTTTCGAACAC[A/G) |
| | TGATAGAAGAGCTGTTGGATGAGGA |
| | (where [A/G] defines the position |
| | of the mutation) |
| 2 | CTGCAGAAAG GCCTGGAATT ACAATCAGAT |
| | GGGCCACATG GCATCCCGGT GAAAGAAAGC |
| | CC |
| | TAACCAGTTT TCTGTCTTGT TTCTGCTTTC |
| | TCCCTACAGT TCCACCAGGT GAGAAGAGTG |
| | ATGACCATCC TTTTCCTTAC TATGGTTATT |
| | TCATACTTTG GTTGCATGAA GGCTGCCCCC |
| | ATGAAAGAAG CAAACATCCG AGGACAAGGT |
| | GGCTTGGCCT ACCCAGGTGT GCGGACCCAT |
| | GGGACTCTGG AGAGCGTGAA TGGGCCCAAG |
| | GCAGGTTCAA GAGGCTTGAC ATCATTGGCT |
| | GACACTTTCG AACAC |
| | R |
| | TGATAGAAGA GCTGTTGGAT GAGGACCAGA |
| | AAGTTCGGCC CAATGAAGAA AACAATAAGG |
| | ACGCAGACTT GTACACGTCC AGGGTGATGC |
| | TCAGTAGTCA AGTGCCTTTG GAGCCTCCTC |
| | TTCTCTTTCT GCTGGAGGAA TACAAAAATT |
| | ACCTAGATGC TGCAAACATG TCCATGAGGG |
| | TCCGGCGCCA CTCTGACCCT GCCCGCCGAG |
| | GGGAGCTGAG CGTGTGTGAC AGTATTAGTG |
| | AGTGGGTAAC GGCGG |
| | CAGACAAAAA GACTGCAGTG GACATGTCGG |
| | GCGGGACGGT CACAGTCCTT GAAAAGGTCC |
| | CTGTATCAAA AGGCCAACTG AAGCAATACT |
| | TCTACGAGAC CAAGTGCAAT CCCATGGGTT |
| | ACACAAAAGA AGGCTGCAGG GGCATAGACA |
| | AAAGGCATTG GAACTCCCAG TGCCGAACTA |
| | CCCAGTCGTA CGTGGGGGCC CTTACCATGG |
| | ATAGCAAAAA GAGAATTGGC TGGCGATTCA |
| | TAAGGATAGA CACTTCTTGT GTATGTACAT |
| | TGACCATTAA AAGGGGAAGA TAGTGGATTT |
| | ATGTTGTATA GATTAGATTA TATTGAGACA |
| | AAAATTATCT ATTTGTATAT ATACATAACA |
| | GGGTAAATTA TTCAGTTAAG AAAAAAATAA |
| | TTTTATGAAC TGCATGTATA AATGAAGTTT |
| | ATACAGTACA GTGGTTCTAC AATCTATTTA |
| | TTGGACATGT CCATGACCAG AAGGGAAACA |
| | GTCATTTGCG CACAACTTAA AAAGTCTGCA |
| | TTACATTCCT TGATAATGTT GTGGTTTGTT |
| | GCCGTTGCCA AGAACTGAAA ACATAAAAAG |
| | TTAAAAAAAA TAATAAATTG CATGCTGCTT |
| | TAATTGTGAA TTGATAATAA ACTGTCCTCT |
| | TTCAGAAAAC AGAAAAAAAC ACACACACAC |
| | ACAACAAAAA TTTGAACCAA AACATTCCGT |
| | TTACATTTTA GACAGTAAGT ATCTTCGTTC |
| | TTGTTAGTAC TATATCTGTT TTACTGCTTT |
| | TAACTECTGA TAGCGTTGGA ATTAAAACAA |
| | TGTCAAGGTG CTGTTGTCAT TGCACCCCCA |
| | AGGGGAACTA ACCGCCTCCC ACACACTATA |
| | TTCCTGCCAC CCCCGCCCCA CCCTACACCG |
| | GCCCCGCACC GCCCC |
| | (wherein R represents the position of the polymorphism) |

1. A method of reducing severity of depression in a human patient having depression, comprising
genetically testing the patient to determine their Val66Met rs6265 polymorphism in the BDNF gene;
identifying the patient as a Val/Val homozygote;
administering an effective amount of ketamine to the patient in an intravenous infusion, and further comprising
administering one or more oral antidepressants.
